(19) United States
(12) Patent Application Publication
(10) Pub. No.: US 2009/0042188 A1
(43) Pub. Date: Feb. 12, 2009

(54) Agent for Differentiating Hematopoietic Stem Cell Into Natural Killer Cell Comprising Vdup1 Protein or Gene Encoding the Same, and a Method of Differentiating Hematopoietic Stem Cell Into Natural Killer Cell Using Thereof

(75) Inventors: Inpyo Choi, Chungcheongnam-do (KR); SukRan Yoon, Taejeon-si (KR); KeeNyung Lee, Taejeon-si (KR); SunWook Kim, Seoul (KR); DaeYul Yoo, Taejeon-si (KR)

Correspondence Address: GREENLEE WINNER AND SULLIVAN P C, 4875 PEARL EAST CIRCLE, SUITE 200, BOULDER, CO, 80301, US

(73) Assignee: Korea Research Institute of Bioscience and Biotechnology, Taejeon-si (KR)

(21) Appl. No.: 11/916,199

(22) PCT Filed: Jun. 8, 2005
(86) PCT No.: PCT/KR2005/001724

§ 371 Date: Nov. 30, 2007

(51) Int. Cl. C12Q 1/68; C07K 14/47; C07H 21/04; C12N 15/63; C12Q 1/02; C12N 15/87; C12N 5/06; A01K 67/027

(52) U.S. Cl. 435/6; 530/350; 536/23.5; 435/320.1; 435/377; 800/18; 435/375; 435/455; 435/29

(57) ABSTRACT

The present invention is related to an agent for differentiating hematopoietic stem cell into natural killer cell comprising VDUP1 protein or gene encoding the same, and a method of differentiating hematopoietic stem cell into natural killer cell using thereof. The present invention reveals for the first time that the VDUP1 gene is a critical factor for the regulation of differentiation of natural killer cell by generating a mouse deficient in VDUP1 gene, which confirms that VDUP1 gene is required for NK maturation. Thus, through the regulation of VDUP1 gene, the modulation of NK cells that have ability to kill cancer cells is possible and can be utilized for cell therapeutics.

### TECHNICAL FIELD

The present invention relates to an agent for differentiating hematopoietic stem cells into natural killer cells and a method of differentiating hematopoietic stem cells into natural killer cells using the same, more precisely, an agent for differentiating hematopoietic stem cells into natural killer cells containing VDUP1 (vitamin D3 upregulating protein 1) or a gene encoding thereof or vitamin D3 regulating VDUP1 as an effective ingredient and a method of differentiating HSCs into NK cells using the same.

### BACKGROUND ART

Hematopoietic stem cells, a kind of stem cells, are able to be differentiated into every blood constituents (red blood cells or erythrocytes, white blood cells or leukocytes, platelets and lymphocytes) by any chance, and are constantly auto-regenerated and differentiated into immune cells in vivo. Among cells forming immune system, natural killer cells (referred as “NK cells” hereinafter) are able to kill non-specific tumor cells. The cytotoxicity of NK cells not only provides a clue for the treatment of a solid tumor by using lymphokine activated killer cell (LAK) and tumor infiltration lymphocytes (TIL) but also is applicable to immunotherapy by donor lymphocyte infusion (J. Immunol., 36: 3910-3915, 1986; Hematologia, 84: 1110-1149, 1999), to develop a new cytotherapy method dealing with rejection reaction after bone marrow transplantation or organ transplantation. The defective differentiation and activation of NK cells are involved in a variety of diseases including breast cancer (Breast Cancer Res. Treat., 66: 255-263, 2003), melanoma (Melanoma Res., 13: 349-356, 2003), lung cancer (lung Cancer, 35: 23-18, 2002), etc, according to recent reports, and thus, new cytotherapy methods have been made to treat such diseases by using NK cells.

NK cells are derived from hematopoietic stem cells in the bone marrow. The NK cell development from HSCs consists of multiple steps, which are not yet completely defined.

Vitamin D3 upregulating protein 1 (VDUP1) was originally reported to be up-regulated by vitamin D3 in HL-60 leukemia cells (Biochem. Biophys. Acta, 1219: 26-32, 1994). It has also been reported recently that VDUP1 interacts with thioredoxin (Trx) to inhibit the activity of Trx and to block the interaction of Trx with other factors (J. Biol. Chem., 274: 21645-21650, 1999, J. Immunol., 164: 6287-6295, 2000). That is, VDUP1 acts as a negative controller of Trx regulating oxidation/reduction in cells, making the cells more sensitive to oxidative stress. In addition, VDUP1 anti sense DNA is reported to be involved in melanin synthesis and tumorigenesis in murine melanoma cells (Immunology Letters, 86: 235-247, 2003) and have anticancer activity by inhibiting cell cycle in tumor cells. In fact, the expression of VDUP1 is reduced in tumor tissues compared with that in normal tissues. VDUP1 expression is dominant in immune cells, but its complete roles in immune cells are not known, yet.

Thus, the present inventors have confirmed that VDUP1 is involved in NK cell differentiation in vitro by regulating IL-2 receptor β (CD122) in VUDP1 knock-out mice and vitamin D3 up-regulating VDUP1 also regulates NK cell differentiation. And, the present inventors completed this invention by confirming the possibility of using a gene regulating differentiation into NK cells which have cytotoxicity and ability to control immune system for cell differentiation and further cancer treatment.

### DISCLOSURE OF INVENTION

### Technical Problem

The present invention confirms the function of VDUP1 (Vitamin D3 upregulating protein 1), a gene regulating differentiation of stem cells into NK cells, and thus provides an agent for NK cell differentiation containing VUDP1 or a gene encoding the protein and vitamin D3 regulating VUDP1 gene and a method for the differentiation using the same.

### Technical Solution

The present invention provides an agent for NK cell differentiation containing VDUP1 protein or a gene encoding the same as an effective ingredient.

The present invention also provides an agent for NK cell differentiation characteristically containing VUDP1 protein represented by SEQ. ID. NO 1.

The present invention provides an agent for NK cell differentiation characteristically containing VDUP1 gene represented by SEQ. ID. No 2.

The present invention provides an agent for NK cell differentiation prepared characteristically by introducing the gene into a non-viral vector or a viral vector.

The present invention provides an agent for NK cell differentiation in which the said non-viral vector is pFLAGmVDUP1 presented in FIG. 18.

The present invention provides an agent for NK cell differentiation including vitamin D3 as an effective ingredient.

The present invention provides an agent for NK cell differentiation which is characterized by regulating VDUP1 gene by vitamin D3.

The present invention provides an agent for NK cell differentiation characteristically available for the treatment of cancer.

The present invention provides an agent for NK cell differentiation to be used for the treatment of cancer characteristically selected from a group consisting of breast cancer, melanoma, stomach cancer and lung cancer.

The present invention also provides a method for differentiating HSCs into NK cells including the step of introducing VDUP1 protein or a gene encoding thereof into HSCs.

The present invention provides a method for differentiating HSCs into NK cells characteristically including the step of co-culture of OP9 stromal cells and IL-15 together.

The present invention provides a method for differentiating HSCs into NK cells characteristically including the step of treating vitamin D3 to HSCs.

The present invention provides a method for differentiating HSCs into NK cells in which the concentration of vitamin D3 is 10-20 nM.

The present invention provides a method for differentiating HSCs into NK cells characteristically including the step of co-culture of OP9 stromal cells and IL-15 together.

The present invention further provides a VDUP1 knock-out mouse showing reduced level of NK cells by the lack of VDUP1 gene.

The present invention provides a VDUP1 knock-out mouse which is characteristically the one deposited with the accession No. of KCTC10794BP.

The present invention also provides a method of elucidating the functions of VDUP1 gene involved in NK cell differentiation by comparing the gene expressions between a wild type mouse and a VDUP1 knock-out mouse.

The present invention further provides a method of increasing cytotoxicity of NK cells characteristically including the step of administrating VDUP1, a gene encoding the protein or vitamin D3.

The present invention provides a method of increasing cytotoxicity of NK cells including the step of administrating IL-2 together with the mentioned factors.

The present invention also provides a VDUP1 expression vector represented by pFLAG-mVDUP1 of FIG. 18.

The present invention further provides a VDUP1 protein as a detection marker for differentiated NK cells or a gene encoding the same.

In the present invention, a “differentiation regulator gene” means a gene regulating the differentiation from stem cells into natural killer cells, more precisely, it means every gene being able to either promote or inhibit the differentiation. That is, a gene of the present invention can promote the differentiation to the next stage, is essential for maintaining each step or inhibiting the differentiation to the next step.

Hereinafter, the present invention is described in detail.

The present invention provides an agent for regulating the NK cell differentiation containing VDUP1 (Vitamin D3 upregulating protein 1) or a gene encoding the protein. The said VDUP1 protein is not limited to a specific one but preferred to be represented by SEQ. ID. No 1. The VUDP1 gene is not limited to a specific one, either but preferred to be represented by SEQ. ID. No 2. The gene is preferred to be included in a non-viral vector or a viral vector, and represented as pFLAG-mVDUP1 of FIG. 18, but not always limited thereto. The present invention also provides an agent for regulating the NK cell differentiation containing vitamin D3 controlling VDUP1 gene as an effective ingredient.

The present inventors separated and purified NK cells and investigated differentiation stage-specific VDUP1 gene expressions, and as a result, the inventors confirmed that VDUP1 gene expression was increased with the maturation of stem cells into NK cells (see FIG. 1). In addition, the activity of CD122 promoter, a gene expressed in NK cells, was elevated by VDUP1 in a dose dependent manner (see FIG. 13). The present inventors proved firstly that VDUP1 gene is a critical factor for the development of NK cells by confirming that the treatment of vitamin D3, known as a regulator of VDUP1 gene, into HSCs induced the differentiation into NK cells (see FIG. 15).

The present invention also provides a VDUP1 knock-out mouse showing the reduced level of NK cells by the lack of VDUP1 (Vitamin D3 upregulating protein 1) gene. The present invention further provides a method of elucidating the functions of VDUP1 (Vitamin D3 upregulating protein 1) gene involved in the NK cell differentiation by comparing the expressions of the genes between a wild type mouse and a VDUP1 (Vitamin D3 upregulating protein 1) knock-out mouse.

The present inventors generated a VUDP1 (Vitamin D3 upregulating protein-1) gene knock-out mouse (see FIG. 2), and deposited the fertilized egg of the mouse at Korean Collection for Type Cultures (KCTC) of Korea Research Institute of Bioscience and Biotechnology on Apr. 26, 2005 (Accession No: KCTC 10794BP).

The number of NK cells was significantly reduced in spleen, bone marrow, and lung of the VDUP1 knock-out mouse, compared with that of a wild type mouse, represented by the expression of NK1.1, a NK marker (see FIG. 5). Single cells were separated from bone marrow and lymph node to investigate the expression of DX-5, another NK marker, using FACS. And as a result, DX-5 expression was decreased, similar to NK1.1 expression, in the VDUP1 knock-out mouse (see FIG. 6). The expressions of NK receptors such as Ly49 NK receptor and NKG2D receptor were also investigated using FACS by double staining with NK1.1-PE and LY49-FITC or NKG2D-FITC. And as a result, the expressions of those NK receptors were reduced or even not induced in lymphocytes of spleen and BM of the VDUP1 knock-out mouse (see FIG. 7). The in vivo expressions of CD122 in small intestines of both a wild type mouse and a VDUP1 knock-out mouse were investigated using FACS. As a result, in vivo expression of CD122 was reduced in small intestine of a VDUP1 knock-out mouse compared with that of wild type mouse (see FIG. 8). The above results confirmed that VUDP1 gene has definitely the function of regulating NK cell differentiation.

The present invention provides a method for differentiating HSCs into NK cells including the step of introducing VDUP1 (Vitamin D3 upregulating protein 1) or a gene encoding the protein into HSCs. The differentiation method is not specifically limited but is preferred to include co-culture of OP9 stromal cells and IL-15. The present invention provides a method for differentiating HSCs into NK cells including the step of administrating vitamin D3 into HSCs. The method is not specifically limited, but is preferred to contain vitamin D3 at the concentration of 10-20 nM, and to include the step of co-culture of OP9 stromal cells and IL-15.

In order to investigate whether or not vitamin D3, which is known to regulate VDUP1, affects directly NK cell differentiation, 1,25-dihydroxy vitamin D(3) was treated to mouse HSCs during its developmental stages through pNK to mNK, and then the treated cells were cultured in the presence of OP9 and IL-15, leading to the development of mNK. Then, FACS analysis and ⁵¹Cr release assay were performed. As a result, NK cell population was increased by vitamin D3 in a dose dependent manner (see FIG. 15). Cytotoxicity to a target cell, Yac-1 of NK cells differentiated by the treatment of low concentration of vitamin D3 (10 nM) was increased, compared with that of vitamin D3 non-treating group (see FIG. 16). In the meantime, mature NK cells were separated from spleen of a wild type mouse, which were treated with IL-2 and vitamin D3 together for 24 hours, followed by ⁵¹Cr-release assay. As a result, it was confirmed that cytotoxicity was increased by the treatment of vitamin D3 (see FIG. 17). The above results indicate that vitamin D3 or VDUP1 is involved in NK cell differentiation and directly affects cytotoxicity therein.

The present invention provides an agent for regulating cell differentiation which is characteristically used for the treatment of cancer. The cancer is not limited to a specific one but is preferably selected from a group consisting of breast cancer, melanoma, stomach cancer and lung cancer.

The defective differentiation and activation of NK cells result in a variety of cancers including breast cancer (Breast Cancer Res Treat., 66: 255-263, 2003), melanoma (Melanoma Res., 2003, 13: 349-356), and lung cancer (Lung Cancer, 35: 23-18, 2002). Therefore, an agent for regulating NK cell differentiation of the present invention can be used for the treatment of cancers by regulating NK cell differentiation.

The agent for regulating cell differentiation of the present invention can be administered orally or parenterally and be used in general forms of pharmaceutical formulation. The agent for regulating cell differentiation of the present invention can be prepared for oral or parenteral administration by mixing with generally used fillers, extenders, binders, wetting agents, disintegrating agents, diluents such as surfactant, or excipients. Solid formulations for oral administration are tablets, pills, dusting powders, granules and capsules. These solid formulations are prepared by mixing one or more suitable excipients such as starch, calcium carbonate, sucrose, lactose, gelatin, etc. Except for the simple excipients, lubricants, for example magnesium stearate, talc, etc, can be used. Liquid formulations for oral administrations are suspensions, solutions, emulsions and syrups, and the formulations mentioned above can contain various excipients such as wetting agents, sweeteners, aromatics and preservatives in addition to generally used simple diluents such as water and liquid paraffin. Formulations for parenteral administration are sterilized aqueous solutions, water-insoluble excipients, suspensions, emulsions, and suppositories. Water insoluble excipients and suspensions can contain, in addition to the active compound or compounds, propylene glycol, polyethylene glycol, vegetable oil like olive oil, injectable ester like ethylolate, etc. Suppositories can contain, in addition to the active compound or compounds, witepsol, macrogol, tween 61, cacao butter, laurin butter, glycerol, gelatin, etc.

The effective dosage of the agent of the present invention is 0.1˜0.2 [symbol] per day and preferably 0.15 [symbol] per day. The frequency of administration is 1˜3 times a day.

### Advantageous Effects

In the present invention, the inventors generated VDUP1 knock-out mice and confirmed through experiments using the mice that VDUP1 is involved in NK cell differentiation in vitro by regulating IL-2 receptor B (CD122) and also vitamin D3 regulating VDUP1 is an important factor for regulating NK cell differentiation. Thus, the gene regulating NK cell differentiation can be effectively used for regulating the differentiation of NK cells having cytotoxicity and functions of controlling immunity and further for the development of anticancer cell therapy.

### BRIEF DESCRIPTION OF THE DRAWINGS

FIG. 1 is an agarose gel photograph. Mouse HSCs were differentiated into mature NK cells (mNK) via NK precursors (pNK) in the presence or absence of OP9 stromal cells (+OP9 or −OP9), and total RNAs were extracted from stage-specific NK developing cells to analyze VUDP1 gene by RT-PCR.

FIG. 2 is a schematic diagram showing the genomic strategy to generate VDUP1 knock-out mice.

FIG. 3 is an agarose gel photograph showing the VDUP1 gene analyzed with VDUP1 knock-out mice (−/−) by RT-PCR.

FIG. 4 is a set of photographs of Northern blotting showing the VDUP1 gene detection in each organs of VDUP1 knock-out mice (−/−).

FIG. 5 is a set of graphs showing the result of FACS analysis. Single cells were isolated from each spleen, bone marrow (BM) and lung of both a wild type mouse (+/+) and a VDUP1 knock-out mouse (−/−), and stained with FITC labeled anti-CD3 antibody and PE labeled anti-NK1.1 antibody. The percentage of NK cells (CD3−/NK1.1+) in lymphocytes was recorded.

FIG. 6 is a set of graphs showing the result of FACS analysis investigating the expression of DX-5, a NK cell marker, in single cells isolated from each bone marrow (BM) and lymph node (LN) of both a wild type mouse (+/+) and a VDUP1 knock-out mouse (−/−).

FIG. 7 is a set of graphs showing the result of FACS analysis investigating the expressions of Ly49 NK receptor and NKG2D receptor in NK cells of spleens and Bone marrows of both a wild type mouse (+/+) and a VDUP1 knock-out mouse (−/−).

FIG. 8 is a set of graphs showing the result of FACS analysis investigating the expression of CD122 in vivo, precisely, in small intestines of both a wild type (+/+) and a VDUP1 knock-out mouse (−/−).

FIG. 9 is a graph showing the cytotoxicity to YAC-1 of spleen cells isolated from both a wild type mouse (+/+) and a VDUP1 knock-out mouse (−/−), and activated with IL-2 for 24 hours.

FIG. 10 is a set of agarose gel photographs. Cytoplasmic DNA was extracted from stage-specific NK developing cells (HSC, pNK, mNK) in vitro from HSCs of a wild type mouse and VDUP1 expressions therein were investigated by RT-PCR.

FIG. 11 is a set of agarose gel photographs. HSCs of both a wild type mouse and a VDUP1 knock-out mouse were differentiated into NK cells in vitro, and the expressions of IL-2 receptor β (CD122), PU.1, ETS-1, LTβR, MEF, id2 and β-actin genes in stage-specific cells (pNK, mNK) were investigated by RT-PCR.

FIG. 12 is a set of graphs showing the result of FACS analysis, by which the expressions of CD122 and NKG2A in stage-specific cells during the differentiation from HSCs into NK cells in vitro were investigated in a wild type mouse and a VDUP1 knock-out mouse.

FIG. 13 is a graph showing the relation of VDUP1 and CD122 which is known as an important factor involved in NK cell differentiation. 293T cells were transfected with CD122luc and pFLAG-mVDUP1 to investigate the relation by luciferase analysis.

FIG. 14 is a set of agarose gel photographs, showing the expressions of IL-15 in bone marrows of both a wild type mouse (+/+) and a VDUP1 knock-out mouse (−/−) investigated by RT-PCR.

FIG. 15 is a set of graphs showing the result of FACS analysis with NK cell group positive for NK1.1. Vitamin D3 was treated during the whole procedure of differentiation from HSCs of a wild type mouse into mNK via pNK, which was progressed by the cell culture in the presence of OP9 and IL-15, resulting in the confirmation of NK cell group positive for NK1.1.

FIG. 16 is a graph showing the cytotoxicity, investigated by ⁵¹Cr release assay, of NK cells for a target cell ‘Yac-1’ developed under the treatment of low concentration of vitamin D3 (10 nM). E/T means the ratio of effective cells to target cells.

FIG. 17 is a graph showing the cytotoxicity of mature NK cells, separated from spleen of a wild type mouse, and then treated with IL-2 and/or vitamin D3 for 24 hours, detected by ⁵¹Cr release assay.

FIG. 18 is a schematic diagram of VDUP1 expression vector.

### BEST MODE FOR CARRYING OUT THE INVENTION

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Example 1

### Isolation of Hematopoietic Stem Cells from Bone Marrow

The bones including tibia and femur of 6-9 week old C57BL/6 mice (Daehan Biolink, Korea) were ground and the ground products were passed through 70- [symbol] cell strainer, to which dissolving solution (Sigma, St. Louse, Mo.) was added to eliminate erythrocytes, resulting in bone marrow cells. The bone marrow cells were reacted with biotin-labeled antibodies specific to system markers (CD11b: macrophage marker, Gr-1: granulocyte marker, B220: B cell marker, NK1.1: NK cell marker, CD2: T cell marker, TER-119: erythrocyte marker), and then washed. The cells were reacted with streptavidin labeled magnetic beads (Miltenyi Biotec, Auburn, Calif.). The magnetic labeled Lin⁺ cells were collected by passing them through CS column (Miltenyi Biotec) within SuperMACS (Miltenyi Biotec, Auburn, Calif.) magnetic field. The Lin⁻ cells passed through the column were reacted with magnetic beads coupled to c-kit. After passing through MS column (Miltenyi Biotec), c-kit⁺ cells remaining on the column were obtained. The purity of Lin⁻ c-kit⁺ hematopoietic stem cells (referred ad HSC cells hereinafter) obtained above was investigated by FACS (BD Bioscience, Mountainview, Calif.), proving over 96% purity.

### Example 2

### Induction of Differentiation from HSCs into NK Cells

HSCs isolated from bone marrow in the above Example 1 were inoculated into 6-well plate (Falcon, USA) using a complete RPMI medium supplemented with mouse SCF (30 ng/ [symbol] , BioSource, Camarillo, Calif.), mouse Flt3L (50 ng/ [symbol] , PeproTech, Rocky Hill, N.J.), mouse IL-7 (0.5 ng/ [symbol] , PeproTech), indomethacin (2 g/ [symbol] , Sigma), gentamycin (20 g/ [symbol] ) and 10% fetal bovine serum, at the concentration of 2×10⁶ cells/well. The cells were cultured in a 37° C., 5% CO₂ incubator for 6 days. Three days later, half of the culture supernatant was discarded and replaced with a fresh new one having the same composition as the above. After further 6 days of culture, CD122⁺ premature NK cells (referred as “pNK cells” hereinafter) were isolated using a FITC-conjugated anti-CD122 and anti-FITC antibody coupled to MACS magnetic beads. The purity of the pNK cells was determined by FACS, confirming over 92% purity.

To generate mature NK cells (referred as “mNK cells” hereinafter), collected HSCs were cultured with or without OP9 stromal cells (Science, 265: 1098-1101, 1994) in the presence of mouse IL-15 (20 ng/ [symbol] , PeproTech, USA). After three days of culture, half of the medium was replaced with a fresh one having the same composition as the earlier. On the 12^{th} day of culture, NK1.1⁺ cells were isolated using a FITC-conjugated anti-NK1.1 antibody and anti-FITC antibody coupled to MACS magnetic beads. The purity of the mature NK cells was determined by flow cytometry (FACS) using anti-CD122, NK1.1, DX-5 and NK cell receptor antibodies.

### Example 3

### Stage-Specific VDUP1 Gene Expressions During Isolated and Purified NK Cell Differentiation

To obtain stage-specific NK developing cells, Lin⁻ c-kit⁺ HSCs (>95%) were isolated from bone marrow of a mouse, which were then cultured in the presence of SCF, Flt-3L, and IL-7 for 6 days. CD122⁺ pNK cells (95%) were isolated, followed by FACS analysis. PNK cells were cultured for another 6 days in the presence of IL-15 only (−OP9) or IL-15 and OP9 stromal cells together (+OP9), followed by FACS analysis. When the cells were cultured together with OP9 stromal cells, the population of mNK cells was more increased (−OP9; 94% and +OP9; >95%). LY49 receptors on mNK cell surface play an important role in mNK cell functioning, and their expressions are regulated by signal transduction by communication with other immune cells. To investigate whether or not the co-culture of bone marrow originated HSCs and stromal cells is essential for the expression of Ly49 receptor, a NK receptor of mNK cells, mNK cells were cultured in the presence of IL-15 only or IL-15 and OP9 stromal cells together. Then, the expressions of Ly49, a NK receptor, were investigated. When the cells were cultured with OP9 stromal cells (+OP9), the expressions of Ly49C/I and Ly49G2, NK receptors, were induced in mNK cells. However, when OP9 stromal cells were not cocultured (−OP9), the expressions of Ly49C/I and Ly49G2 were not induced. The results indicate that the co-culture with OP9 stromal cells is essential for the maturation of NK cells.

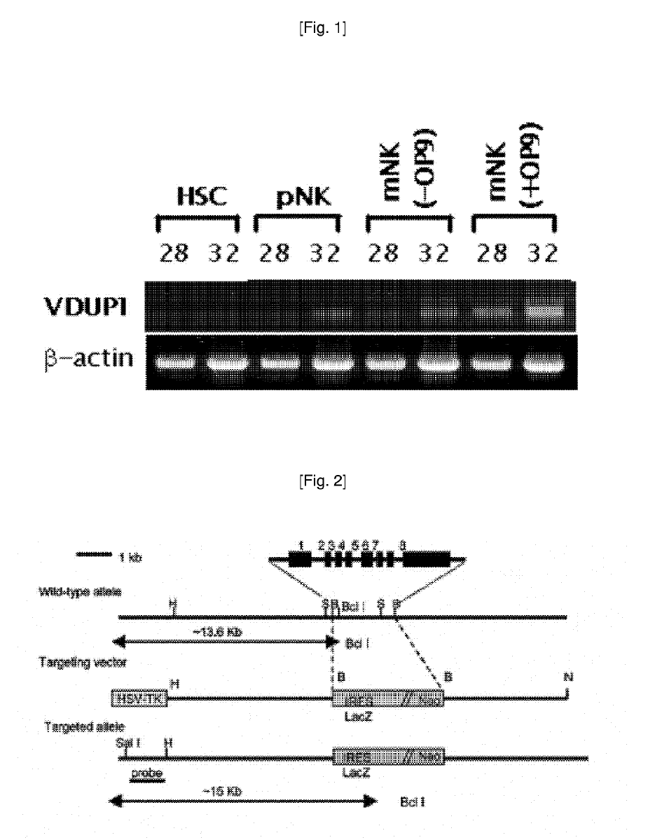

NK cell differentiation stage-specific VDUP1 gene expressions were investigated by RT-PCR (FIG. 1). RNAs were extracted from all cells by using Trizol reagent (Life Technology, USA) according to the manufacturer's instruction, and cDNAs were synthesized by using RT-PCR kit (Quiagen, Germany) according to the manufacturer's instruction. The PCR mixture containing cDNA was heated at 95° C. for 1 minute, and amplifications were performed with HSCs and mNK cells as follows; 28 cycles or 32 cycles of 95° C./1 minute, 55° C./1 minute, and 72° C./2 minutes. PCR with pNK cells was performed with 32 cycles of 95° C./1 minute, 60° C./1 minute, 72° C./2 minutes, and followed by extension at 72° C. for 10 minutes. The PCR products were electrophoresed and stained with ethidium bromide.

The result of RT-PCR investigating NK cell differentiation stage-specific VDUP1 gene expressions was shown in FIG. 1. As shown in FIG. 1, the expression of VDUP1 gene was increased stage dependently, namely as NK cells being matured.

### Example 4

### Generation of VDUP1 Knock-Out Mice

To produce VDUP1 knock-out mice, a targeting vector in which the sequence from exon 1 to exon 8 of VDUP1 gene was replaced with the lacZ/neo cassette gene was constructed (FIG. 2). The vector was introduced into 129Sv mouse embryonic stem cells, which were cultured in medium supplemented with G418 antibiotics and gancyclovir. Among live embryonic cells on the medium, those showing mutation in one allele of the VDUP1 gene were selected and injected into the blastocyst embryos of C57BL/6 mice to produce chimeric mice. The mutant allele was successfully transmitted to the next generation via their germ line. So, VDUP1 knock-out mice were generated.

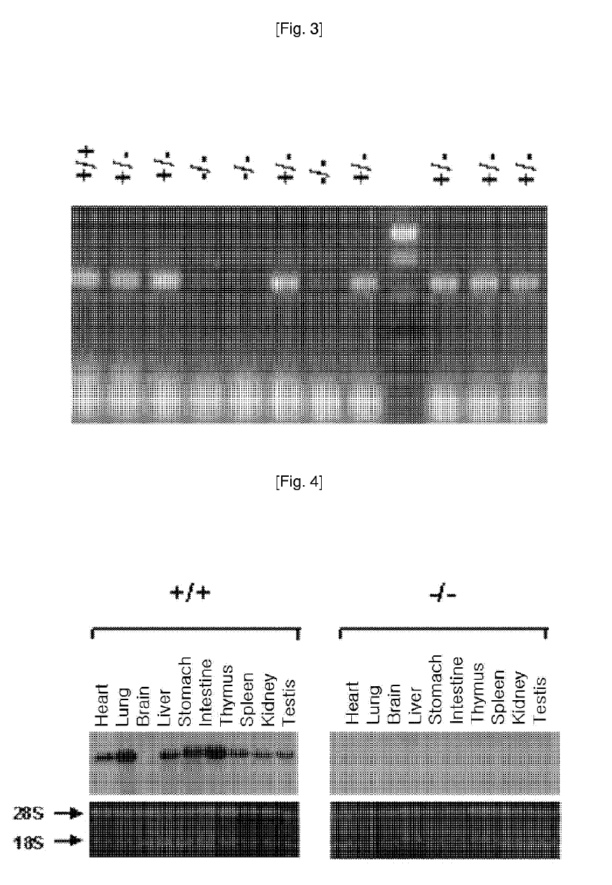

The successful generation of VDUP1 knock-out mice was confirmed by RT-PCR and Northern blotting with each organ (FIG. 3 and FIG. 4). For RT-PCR, cDNA was obtained as indicated in Example 3, and the primers of 5′-ATTCCCCTTCCAGGTGGA-3′ and 5′-TTGAAATTGGCTCTGT-3′ were used. Precisely, PCR mixture containing cDNA was heated at 95° C. for 1 minute, followed by 32 cycles of 95° C./1 minute, 55° C./1 minute, and 72° C./2 minutes, and final extension was induced at 72° C. for 10 minutes. The amplified PCR product was electrophoresed and stained with ethidium bromide. For Northern blotting, organs including stomach, brain, lung and spleen, were taken from a mouse. They were homogenized in RNAzol B solution (Tel-Test, Friendswood, Tex.) by using a tissue homogenizer, and the resultant RNAs were purified. 30 [symbol] of RNA was electrophoresed in 1% agarose gel containing 2.2 M formaldehyde, and then adhered onto nylon membrane (GeneScreen^{PLUS}, NEN Life Science Products, Boston, Mass.). The nylon membrane was reacted in ExpressHyb solution (Clontech, USA) containing ³²P-labeled VDUP1 cDNA at 65° C. for 16 hours. The membrane was washed more than three times to eliminate unspecifically loaded probe, followed by autoradiography.

As shown in FIG. 3 and FIG. 4, VDUP1 gene was not detected at all in VDUP1 knock-out mice (−/−), proving successful elimination of the gene.

The present inventors deposited the fertilized egg of the VDUP1 knock-out mouse at Korean Collection for Type Cultures (KCTC) of Korea Research Institute of Bioscience and Biotechnology on Apr. 26, 2005 (Accession No: KCTC 10794BP).

### Example 5

### NK Cell Differentiation in VDUP1 Knock-Out Mice

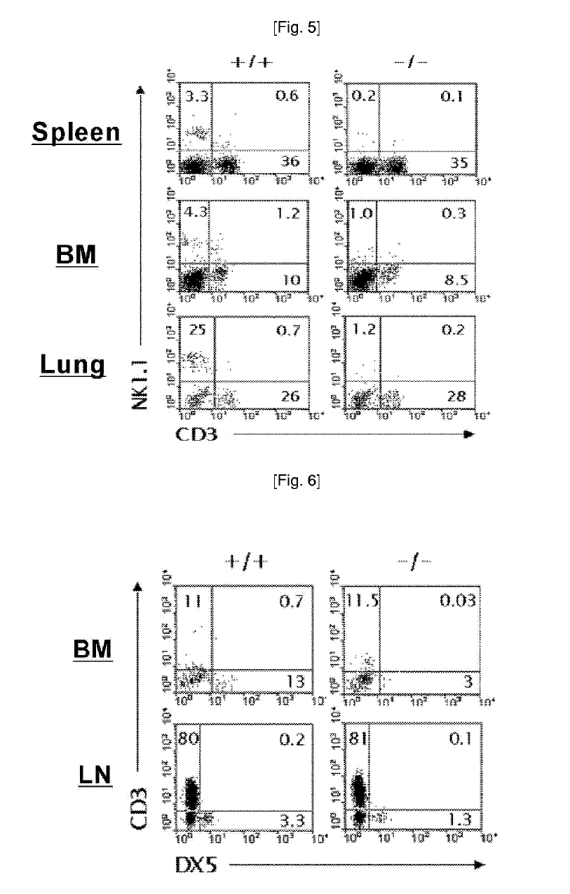

Single cells were isolated from spleen, bone marrow, and lung of both a wild type mouse and a VDUP1 knock-out mouse, which were stained with FITC-labeled anti-CD3 antibody and PE-labeled anti-NK1.1 antibody to determine the percentage of NK cells (CD3−/NK1.1+) in lymphocytes by FACS analysis (FIG. 5).

As shown in FIG. 5, NK cell population was remarkably reduced in spleen, BM, and lung of a VDUP1 knock-out mouse, compared with a wild type mouse.

Single cells were also isolated from bone marrow and lymph node and the expression of another NK marker DX-5 therein was investigated by FACS analysis (FIG. 6).

As shown in FIG. 6, in accordance with the reduced expression of NK1.1, the expression of another NK marker DX-5 was reduced in a VDUP1 knock-out mouse.

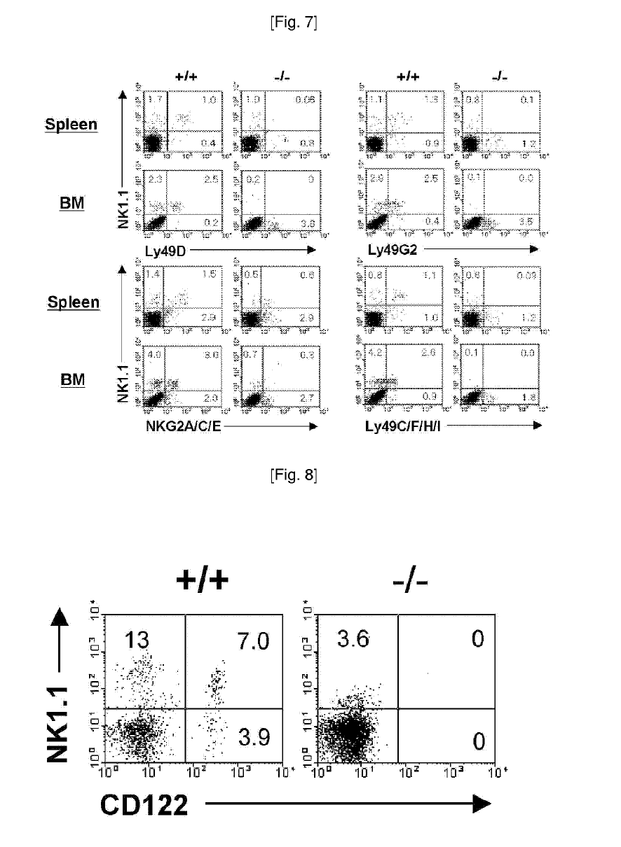

Double staining with NK1.1-PE and Ly49-FITC or NKG2D-FITC was performed, followed by FACS analysis to investigate the expressions of NK receptors ‘Ly49 NK receptor and NKG2D-FITC receptor’ in NK cells (FIG. 7).

As shown in FIG. 7, the expressions of NK receptors were also reduced or not even induced in spleen and bone marrow of a VDUP1 knock-out mouse.

The in vivo expression of CD122 in small intestine was compared by FACS between a wild type mouse and a VDUP1 knock-out mouse (FIG. 8).

As shown in FIG. 8, the in vivo expression of CD122 in small intestine was not induced in a VDUP1 knock-out mouse.

### Example 6

### NK Cytotoxicity Assay

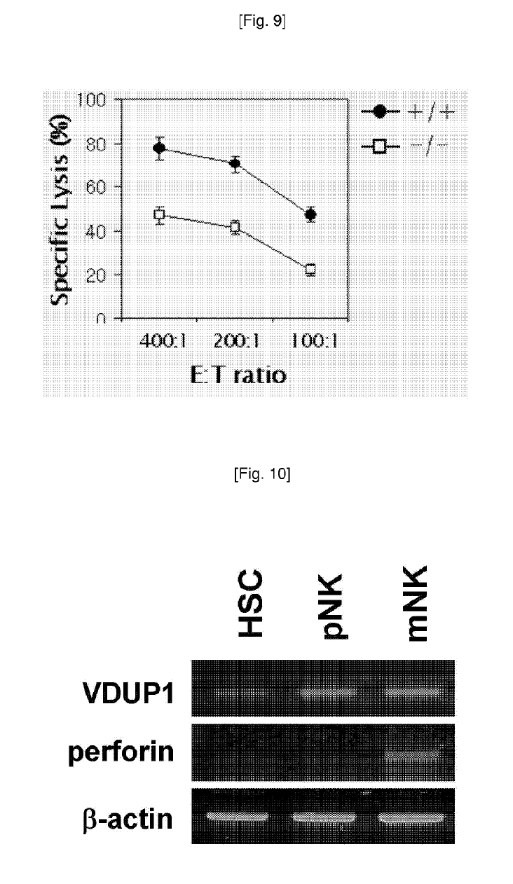

NK cells, differentiated in vitro or isolated from spleen, were treated with IL-2 (10 u/ml), followed by culture for 24 hours. After being washed, NK cells were plated, according to ratios of effector cells to target cells, into a 96 well plate (well round bottom plate, Falcon, USA) containing target cells (⁵¹Cr-labeled Yac-1 cells, 10⁴/well), followed by further culture for 4 hours. Upon completion of the culture, radioactivity of 100 ul of supernatant was measured by γ counter (FIG. 9).

As shown in FIG. 9, cells isolated from spleen of each wild type mouse and VDUP1 knock-out mouse were activated by IL-2 for 24 hours and then NK mediated cytotoxicity for YAC-1 was measured. As a result, cytotoxicity was remarkably reduced in a VDUP1 knock-out mouse, compared with that in a wild type mouse.

### Example 7

### Investigation of a Gene Expressed During NK Cell Development

To investigate the effect of VDUP1 on NK cell development, cytoplasmic RNAs were extracted from each stage of in vivo NK development from HSCs of a wild type mouse as described previously in Example 3. Then, RT-PCR with VDUP1 was performed (FIG. 10).

As shown in FIG. 10, the expression of VDUP1 began to increase from the stage of pNK cells and continued to the stage of mNK cells.

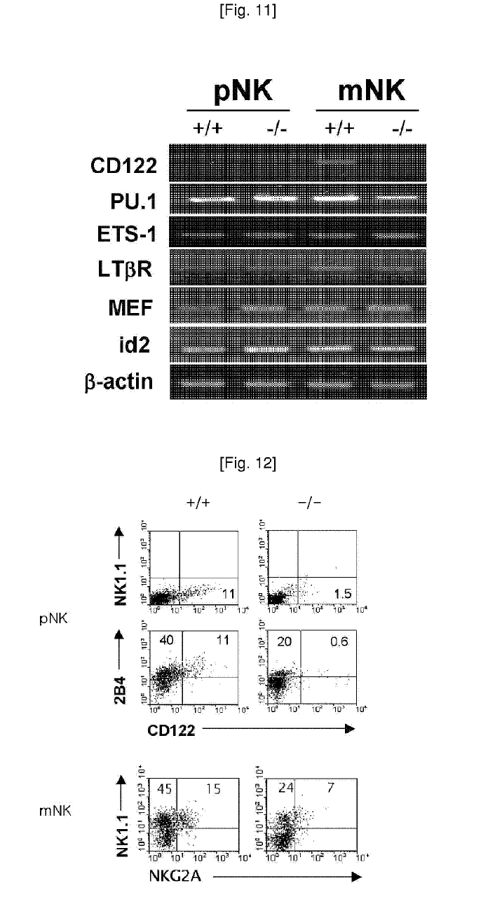

In the meantime, in vitro NK cell differentiation was induced from HSCs of both a wild type mouse and a VDUP1 knock-out mouse, and the stage-specific expressions of CD122, PU.1, ETS-1, LtbetaR, MEF, ld2, and β-actin genes were compared by RT-PCR (FIG. 11).

As shown in FIG. 11, the expression of CD122 was reduced in pNK and mNK cells of a VDUP1 knock-out mouse, compared with that in a wild type mouse.

In addition, stage-specific expressions of CD122 and NK1.1 were investigated in vitro by FACS (FIG. 12).

As shown in FIG. 12, the expression of CD122 was decreased in pNK cells of a VDUP1 knock-out mouse, compared with that in a wild type mouse. The expressions of NK1.1 and NKG2A in mNK cells were also decreased.

### Example 8

### The Effect of VDUP1 on CD122 Promoter Activity

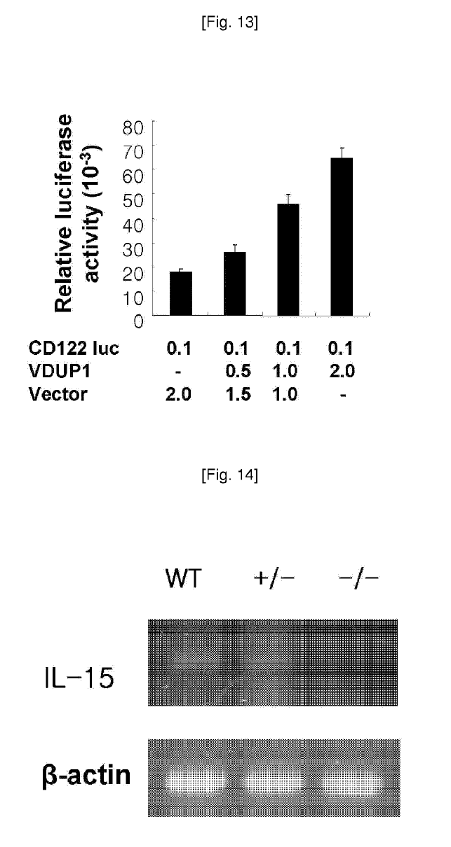

293T cells were transfected with 0.1 [symbol] of CD122 promoter (−857/97) luciferase reporter plasmid, 0.1 [symbol] of renilla luciferase plasmid (CD122luc) and different concentrations of VDUP1 expression vector (pFLAG-mVDUP1; VDUP1 expression vector was constructed by inserting mouse VDUP1 cDNA into the sites of Hind III and Xba I of pFLAG-CMV2 expression vector (Clontech, USA) (FIG. 18)). The concentration of total DNA was adjusted by using empty vector. After 48 hours of culture, luciferase activity was measured using cell lysate according to the manufacturer's instruction (Promega, Madison, Wis.) (FIG. 13). Transfection efficiency was standardized by counting renilla luciferase activity.

As shown in FIG. 13, CD122 promoter activity was promoted VDUP1 dose-dependently.

### Example 9

### The Effect of Vitamin D3 on NK Cell Development

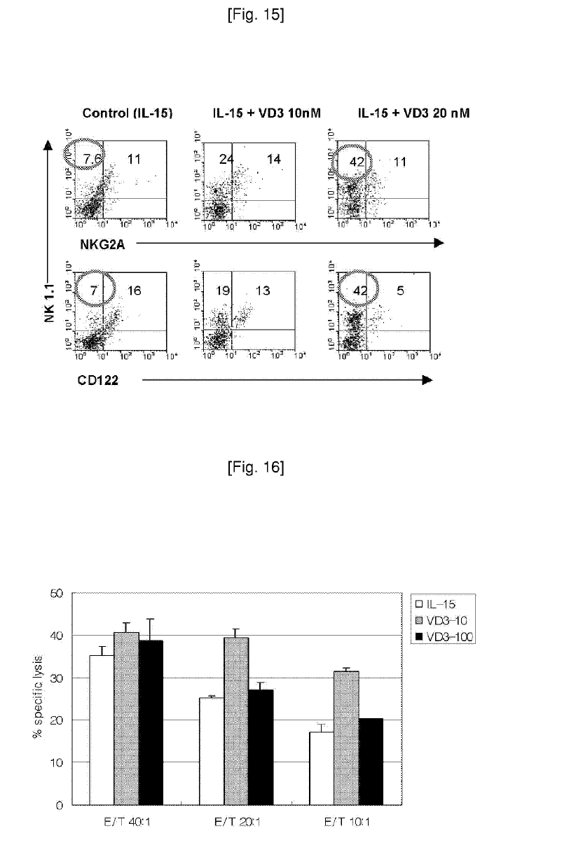

To investigate the effect of vitamin D3, known to regulate VDUP1, on NK cell development, 1,25-dihydroxy vitamin D(3) was treated to each stage of differentiation from mouse HSCs into mNK cells via pNK. The cells were matured by the culture in the presence of OP9 and IL-15, then FACS analysis (FIG. 15) and ⁵¹Cr release assay (FIG. 16) were performed.

As shown in FIG. 15, NK cell population was increased by vitamin D3 in a dose dependent manner.

As shown in FIG. 16, NK mediated cytotoxicity for Yac-1 was increased in those cells treated with low concentration (10 nM) of vitamin D3, compared with vitamin D3 non-treating group.

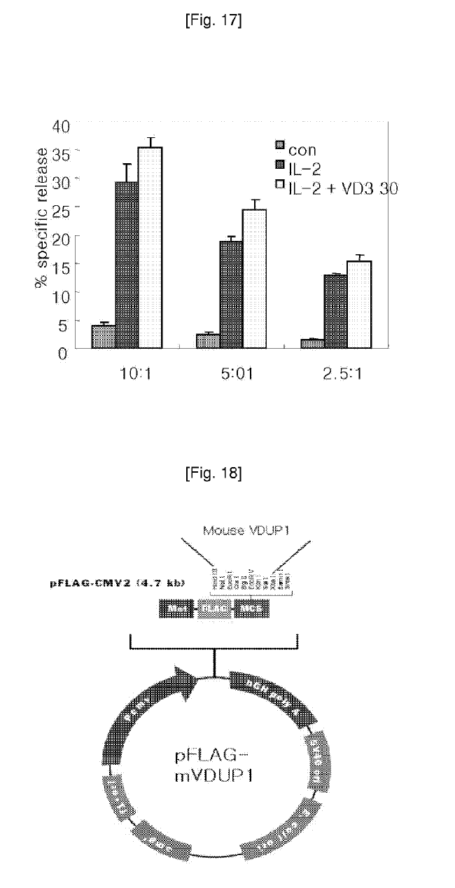

Further, mature NK cells were isolated from spleen of a wild type mouse. The cells were treated with vitamin D3 together with IL-2 for 24 hours, followed by ⁵¹Cr release assay (FIG. 17).

As shown in FIG. 17, NK mediated cytotoxicity was increased with the treatment of vitamin D3.

The above results indicate that vitamin D3 or VDUP1 is involved in NK cell development and directly affects cytotoxicity.

### INDUSTRIAL APPLICABILITY

As explained hereinbefore, the present inventors generated VDUP1 knock-out mice and confirmed through experiments using the mice that VDUP1 is involved in NK cell differentiation in vitro by regulating IL-2 receptor B (CD122) and also vitamin D3 regulating VDUP1 is an important factor for regulating NK cell differentiation. Thus, the gene regulating NK cell differentiation can be effectively used for regulating the differentiation of NK cells that have cytotoxicity and functions of controlling immunity and further for the development of anticancer cell therapy.

### SEQUENCE LISTING

SEQ. ID. No 1 is an amino acid sequence of mouse VDUP1 protein.

SEQ. ID. No 2 is a nucleotide sequence of mouse VDUP1 gene.

A sequence listing of the sequences described above was already filed to the Receiving Office when the present application was filed.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. An agent for NK cell differentiation containing VDUP1 (Vitamin D3 upregulating protein 1) or a gene encoding the protein as an effective ingredient.

2. The agent for NK cell differentiation as set forth in claim 1, wherein the VDUP1 (Vitamin D3 upregulating protein 1) is represented by SEQ. ID. No 1.

3. The agent for NK cell differentiation as set forth in claim 1, wherein the VDUP1 (Vitamin D3 upregulating protein 1) gene is represented by SEQ. 1D. No 2.

4. The agent for NK cell differentiation as set forth in claim 1, wherein the gene is included in a non-viral vector or a viral vector.

5. The agent for NK cell differentiation as set forth in claim 4, wherein the non-viral vector is pFLAG-mVDUP1 represented in FIG. 18.

6. An agent for NK cell differentiation containing vitamin D3 as an effective ingredient.

7. The agent for NK cell differentiation as set forth in claim 6, wherein the vitamin D3 regulates VDUP1 (Vitamin D3 upregulating protein 1) gene.

8. The agent for NK cell differentiation as set forth in of claims 1 to 7, wherein the agent is used for anticancer cell therapy.

9. The agent for NK cell differentiation as set forth in claim 8, wherein the cancer is selected from a group consisting of breast cancer, melanoma, stomach cancer, hepatoma and lung cancer.

10. A method for differentiating hematopoietic stem cells (HSCs) into NK cells including the step of introducing VDUP1 (Vitamin D3 upregulating protein 1) or a gene encoding thereof into HSCs.

11. The method for differentiating hematopoietic stem cells (HSCs) into NK cells as set forth in claim 10, wherein the HSCs are cultured with OP9 stromal cells in the presence of IL-15.

12. A method for differentiating hematopoietic stem cells (HSCs) into NK cells including the step of administrating vitamin D3 into HSCs.

13. The method for differentiating hematopoietic stem cells (HSCs) into NK cells as set forth in claim 12, wherein the concentration of vitamin D3 is 10-20 nM.

14. The method for differentiating hematopoietic stem cells (HSCs) into NK cells as set forth in claim 12, wherein the HSC cells are cultured with OP9 stromal cells in the presence of IL-15.

15. A VDUP1 (Vitamin D3 upregulating protein 1) knock-out mouse showing reduced level of NK cells owing to the lack of VDUP1 (Vitamin D3 upregulating protein 1) gene.

16. The VDUP1 (Vitamin D3 upregulating protein 1) knock-out mouse as set forth in claim 15, wherein the mouse is deposited with the accession No. of KCTC10794BP.

17. The method of elucidating the functions of VDUP1 (Vitamin D3 upregulating protein 1) gene involved in NK cell differentiation by comparing the gene expressions between a wild type mouse and a VDUP1 (Vitamin D3 upregulating protein 1) knock-out mouse.

18. A method of increasing cytotoxicity of NK cells including the step of administrating VDUP1 (Vitamin D3 upregulating protein 1), a gene encoding the protein or vitamin D3.

19. The method of increasing cytotoxicity of NK cells as set forth in claim 18, wherein the step of administrating IL-2 is also included.

20. A VDUP1 expression vector represented by pFLAG-mVDUP1 in FIG. 18.

21. A method for predicting differentiation stage of NK cells comprising measuring the expression levels of VDUP1 (Vitamin D3 upregulating protein 1).


SEQUENCE LISTING (2 sequences)

SEQ ID NO: 1 - protein, 396 aa, Mus musculus
MVMFKKIKSFEVVFNDPEKVYGSGEKVAGRVIVEVCEVTRVKAVRILACGVAKVLWMQGSQQCKQTLDYLRYEDTLLLEEQPTGENEMVIMRPGNKYEYKFGFELPQGPLGTSFKGKYGCVDYWVKAFLDRPSQPTQEAKKNFEVMDLVDVNTPDLMAPVSAKKEKKVSCMFIPDGRVSVSARIDRKGFCEGDDISIHADFENTCSRIVVPKAAIVARHTYLANGQTKVFTQKLSSVRGNHIISGTCASWRGKSLRVQKIRPSILGCNILKVEYSLLIYVSVPGSKKVILDLPLVIGSRSGLSSRTFSMASRTSSEMSWIDLNIPDTPEAPPCYMDIIPEDHRLESPTTPLLDDVDDSQDSPIFMYAPEFQFMPPPTYTEVDPCVLNNNNNNNNVQ

SEQ ID NO: 2 - DNA, 1191 bp, Mus musculus
atggtgatgttcaagaagatcaagtcttttgaggtggtcttcaacgaccccgagaaggtgtacggcagcggggagaaggtggccggacgggtaatagtggaagtgtgtgaagttacccgagtcaaagccgtcaggatcctggcttgcggcgtggccaaggtcctgtggatgcaagggtctcagcagtgcaaacagactttggactacttgcgctatgaagacacacttctcctagaagagcagcctacaggtgagaacgagatggtgatcatgaggcctggaaacaaatatgagtacaagttcggcttcgagcttcctcaagggcccctgggaacatcctttaaaggaaaatatggttgcgtagactactgggtgaaggcttttctcgatcgtcccagccagccaactcaagaggcaaagaaaaacttcgaagtgatggatctagtggatgtcaatacccctgacctaatggcaccagtgtctgccaaaaaggagaagaaagtttcctgcatgttcattcctgatggacgtgtgtcagtctctgctcgaattgacagaaaaggattctgtgaaggtgatgacatctccatccatgctgactttgagaacacgtgttcccgaatcgtggtccccaaagcggctattgtggcccgacacacttaccttgccaatggccagaccaaagtgttcactcagaagctgtcctcagtcagaggcaatcacattatctcagggacttgcgcatcgtggcgtggcaagagcctcagagtgcagaagatcagaccatccatcctgggctgcaacatcctcaaagtcgaatactccttgctgatctacgtcagtgtccctggctccaagaaagtcatccttgatctgcccctagtgattggcagcaggtctggtctgagcagccggacattcagcatggccagccggacgagctctgagatgagctggatagacctaaacatcccagataccccagaagctcctccttgctatatggacatcattcctgaagatcacagactagagagccccaccacccctctgctggacgatgtggacgactctcaagacagtcctatctttatgtacgcccctgagttccagttcatgcccccacccacttacactgaggtggatccgtgcgtccttaacaacaacaacaacaacaacaacgtgcagtga